(19) United States
(12) Patent Application Publication
(10) Pub. No.: US 2007/0014778 A1
(43) Pub. Date: Jan. 18, 2007

(54) Endoribonuclease and uses thereof

(76) Inventor: Chow Lee, Prince George (CA)

Correspondence Address: Ralph A. Dowell of DOWELL & DOWELL P.C., 2111 Eisenhower Ave, Suite 406, Alexandria, VA, 22314, US

(21) Appl. No.: 11/446,524

(22) Filed: Jun. 5, 2006

Related U.S. Application Data
(60) Provisional application No. 60/673,775, filed on Jun. 6, 2005.

(51) Int. Cl. A61K 48/00; C12Q 1/68; C07H 21/02; C12P 21/06; A61K 38/46; C12N 9/22

(52) U.S. Cl. 424/94.6; 514/44; 435/199; 435/6; 435/69.1; 435/320.1; 435/325; 536/23.1

(57) ABSTRACT

The invention provides in part a substantially pure endoribonuclease, having a molecular weight of about 34-48 KDa on a 12% denaturing sodium dodecyl sulfate polyacrylamide gel and capable of preferentially cleaving a RNA molecule at sites 3′ of uracil residues or sites 5′ of adenine residues. The invention provides methods of purification of the endoribonuclease, together with uses of the endoribonuclease.

### FIELD OF THE INVENTION

The invention relates to ribonucleases. More specifically, the invention provides a novel endoribonuclease activity and uses thereof.

### BACKGROUND OF THE INVENTION

Ribonucleases (RNases) are enzymes that catalyze the hydrolysis of ribonucleic acid (RNA) by cleaving phosphodiester bonds, generating free 5′-hydroxyl or free 3′-phosphate groups. Although many ribonucleases have affinity for DNA, in general hydrolysis does not occur due to the absence of the 2′-hydroxyl group in DNA that is required for the formation of the cleavage intermediate. An endoribonuclease hydrolyzes the interior bonds of RNA molecules, generating oligonucleotides and polynucleotides, whereas an exoribonuclease hydrolyzes the terminal bonds of RNA molecules, producing mononucleotides.

RNases have been implicated as playing an important role in the maturation and maintenance of cellular RNA (e.g., ribosomal RNA, transfer RNA, messenger RNA) in cells. Many ribonucleases have been implicated as having other activities, in addition to their ribonuclease activity. These activities include anti-viral, anti-neoplastic, anti-bacterial, anti-parasitic, and neurotoxic activities. For example, eosinophil cationic protein (ECP) is a ribonuclease secreted by activated human eosinophils that has anti-parasitic, antibacterial, and neurotoxic activities (Rosenberg H F, J Biol Chem., 270(14):7876-81, 1995), and a frog ribonuclease termed “onconase” has been shown to have antiviral and antineoplastic activities (Ardelt et al., J Biol Chem., 5:266(1):245-51, 1991; Saxena et al., J Biol Chem., 271(34):20783-8, 1996).

The most well-characterized RNases have been isolated from bacteria, and there are relatively few well-characterized eukaryotic, e.g., mammalian ribonucleases, particularly those that degrade mature mRNAs (Guhaniyogi and Brewer, Gene 265:11-23, 2001).

### SUMMARY OF THE INVENTION

In one aspect, the invention provides a substantially pure endoribonuclease, where the endoribonuclease has a molecular weight of about 34 to about 48 KDa on a 12% denaturing SDS polyacrylamide gel and is capable of preferentially cleaving a RNA molecule at sites 3′ of uracil residues or sites 5′ of adenine residues.

The endoribonuclease may have any one or more of the following properties: thermostable to a temperature of about 70° C.; active between a temperature of about 4° C. to about 60° C.; inactive in the presence of Cu²⁺; active in the presence of Mg²⁺, Ca²⁺, Zn²⁺ or Ni²⁺; active in the presence of RNAase inhibitors such as vanadyl ribonucleoside complex (VRC), RNasin, RNaseOUT, Protector RNase Inhibitor, or Anti-RNase; capable of cleaving a RNA molecule at sites 3′ of cytosine residues or sites 5′ of guanine residues; capable of cleaving non-base paired RNA, single stranded RNA, or double stranded RNA; capable of cleaving a UA, CA, UG, or CU sequence; and/or capable of cleaving c-myc mRNA or SARS mRNA. The c-myc mRNA may include the CRD sequence and the SARS mRNA may include orf3, orf1b, or spike sequences. The endoribonuclease may be a mammalian endoribonuclease, for example, a rat or human endoribonuclease.

In other aspects, the invention provides methods for treating or preventing a disorder associated with aberrant RNA expression, viral RNA expression, or c-myc RNA expression, by administering an endoribonuclease according to the invention. The viral RNA may be SARS orf3, SARS spike, or SARS orf1b: the c-myc RNA may be CRD RNA. The endoribonuclease may include a fusion polypeptide, which may include a targeting molecule capable of targeting the endoribonuclease to a virus-infected cell or a cancer cell. The endoribonuclease may be provided together with a pharmaceutically acceptable carrier.

In other aspects, the invention provides a method of cleaving a RNA molecule by contacting the RNA molecule with the endoribonuclease under conditions in which the endoribonuclease is active, where the endoribonuclease cleaves the RNA molecule. The RNA molecule may be non-base paired RNA, single stranded RNA, or double stranded RNA, or may be a c-myc RNA molecule or a SARS RNA molecule (e.g., orf3, spike, or orf1b). The RNA molecule may include a UA, CA, UG, or CU sequence or other cleavage sequence described herein.

In other aspects, the invention provides a method of screening a compound for modulating endoribonuclease activity by a) providing the endoribonuclease; b) providing a RNA molecule substrate capable of being cleaved by the endoribonuclease; c) providing a test compound; and d) determining whether the test compound modulates the cleavage of the RNA molecule substrate. The method may be carried out within a cell or carried out using a cell free system. The RNA molecule substrate may be provided in a sample.

In other aspects, the invention provides a kit including the endoribonuclease of the invention (e.g. lyophilized or in a suitable buffer in a container), together with instructions for performing an endonuclease assay or for administering to a subject. The kit may include suitable buffers and other components for performing an endoribonuclease assay. The kit may include a targeting molecule and compounds for linking the targeting molecule to the endoribonuclease.

A compound is “substantially pure” when it is separated from the components that naturally accompany it. Typically, a compound is substantially pure when it is at least 10%, 20%, 30%, 40%, 50%, or 60%, more generally 70%, 75%, 80%, or 85%, or over 90%, 95%, or 99% by weight, of the total material in a sample. In some embodiments, a compound is substantially pure when it is at least 100 fold, 200 fold, 300 fold, 400 fold, 500 fold, 600 fold, 700 fold, or over 800 fold pure relative to a starting material (e.g., rat liver) from which the compound is purified. Thus, for example, a polypeptide that is chemically synthesized, isolated by known purification techniques, or isolated as described herein will be generally be substantially free from its naturally associated components. A substantially pure compound can be obtained, for example, by extraction from a natural source or by chemical synthesis. Purity can be measured using any appropriate method such as column chromatography, gel electrophoresis, HPLC, etc. In some instances, the isolated material will form part of a composition (for example, a crude extract containing other substances), buffer system or reagent mix. In other circumstance, the material may be purified to essential homogeneity, for example as determined by sodium dodecyl sulfate polyacrylamide gel electrophoresis (SDS-PAGE) or column chromatography such as HPLC.

An “endoribonuclease” or “endonuclease,” as used herein, is a polypeptide that is capable of cleaving the interior bonds of RNA molecules, generating oligonucleotides and polynucleotides. The RNA molecules may be single-stranded RNA molecules, or may be double-stranded RNA molecules. The RNA molecules may be double-stranded as a result of hybridization with a complementary RNA molecule (e.g., antisense RNA molecule) or as a result of folding of a single-stranded RNA molecule into double-stranded “stem” regions.

“Thermostable” as used herein means the ability to hydrolyze the cleavage of a RNA molecule at any given temperature, when incubated for at least ten (10) minutes.

“Activity” as used herein refers to the ability of the endoribonuclease of the invention to catalyze the hydrolysis of a substrate RNA molecule under standard conditions of, for example, time, temperature, metal ion concentration, or pH, for example, at 37° C. for 10 minutes in a standard 20-25 μl reaction mixture which includes 2 mM Dithiothreitol, I unit of RNasin (Promega), 2 mM magnesium acetate, 50 mM potassium acetate, 0.1 mM spermidine, and 10 mM Tris-HCl pH 7.6. The activity or inactivity of the endoribonuclease is measured relative to these standard conditions, and may vary by at least 10%, 20%, 30%, 40%, 50%, 60%, 70%, 80%, or 90%, or by over 100% relative to the standard conditions. The exact amount of variation in activity is not critical, as long as it is statistically significant, as measured by known statistical methods.

By “preferentially cleaves” is meant that the endoribonuclease of the invention cleaves a RNA molecule at sites 3′ of uracil residues or sites 5′ of adenine residues at least 10%, 20%, 30%, 40%, 50%, 60%, 70%, 80%, or 90%, or over 100% more frequently or efficiently under standard conditions relative to any other possible cleavage site. In some embodiments, “preferentially cleaves” means that the endoribonuclease of the invention cleaves a single-stranded RNA molecule at sites between 3′ of uracil residues and 5′ of adenine residues (U-A); sites between 3′ of uracil residues and 5′ of guanine residues (U-G); sites between 3′ of cytosine residues and 5′ of uracil residues (C-U); and/or sites between 3′ of cytosine residues and 5′ of adenine residues (C-A) at least 10%, 20%, 30%, 40%, 50%, 60%, 70%, 80%, or 90%, or over 100% more frequently or efficiently under standard conditions relative to any other possible cleavage site. In some embodiments, “preferentially cleaves” means that the endoribonuclease of the invention cleaves a single-stranded or double-stranded RNA molecule at U-A, C-A, or U-G sites at least 10%, 20%, 30%, 40%, 50%, 60%, 70%, 80%, or 90%, or over 100% more frequently or efficiently under standard conditions relative to any other possible cleavage site. In some embodiments, “preferentially cleaves” means that the endoribonuclease of the invention cleaves a single-stranded RNA molecule at a C-U site at least 10%, 20%, 30%, 40%, 50%, 60%, 70%, 80%, or 90%, or over 100% more frequently or efficiently under standard conditions relative to any other possible cleavage site. In some embodiments, “preferentially cleaves” means that the endoribonuclease of the invention cleaves a single-stranded RNA molecule at least 10%, 20%, 30%, 40%, 50%, 60%, 70%, 80%, or 90%, or over 100% more frequently or efficiently under standard conditions relative to a double-stranded RNA molecule. In some embodiments, “preferentially cleaves” means that the endoribonuclease of the invention cleaves a double-stranded stem region of a folded single-stranded RNA molecule at least 10%, 20%, 30%, 40%, 50%, 60%, 70%, 80%, or 90%, or over 100% more frequently or efficiently under standard conditions relative to a hybridized double-stranded RNA molecule. The exact amount of cleavage is not critical, as long as it is statistically significant, as measured by known statistical methods.

A “targeting moiety” is a compound (e.g, nucleic acid, polypeptide, small molecule) that is capable of specifically binding a target cell or molecule, for example, a cancer cell or molecule, a viral cell or molecule, or a specific mRNA molecule. In some embodiments, a targeting moiety may include part of a fusion protein that includes a compound according to the invention.

“Modulating” or “modulates” means changing, by either increase or decrease. The increase or decrease may be a change of any number value between 10% and 90%, or of any number value between 30% and 60%, or may be over 100%, when compared with a control or reference sample or compound.

A “test compound” is any naturally-occurring or artificially-derived chemical compound. Test compounds may include, without limitation, peptides, polypeptides, synthesized organic molecules, naturally occurring organic molecules, and nucleic acid molecules. A test compound can “compete” with a known compound such as a endoribonuclease by, for example, interfering with endoribonuclease activity or by interfering with any biological response induced by the known compound.

Generally, a test compound can exhibit any value between 10% and 200%, or over 500%, modulation when compared to a endoribonuclease or other reference compound. For example, a test compound may exhibit at least any positive or negative number from 10% to 200% modulation, or at least any positive or negative number from 30% to 150% modulation, or at least any positive or negative number from 60% to 100% modulation, or any positive or negative number over 100% modulation. A compound that is a negative modulator will in general decrease modulation relative to a known compound, while a compound that is a positive modulator will in general increase modulation relative to a known compound. The exact amount of modulation is not critical, as long as it is statistically significant, as measured by known statistical methods.

A “sample” can be any organ, tissue, cell, or cell extract isolated from a subject, such as a sample isolated from a mammal having a cell proliferative disorder, a viral disorder, or an aberrant RNA expression disorder. For example, a sample can include, without limitation, tissue (e.g., from a biopsy or autopsy), cells, peripheral blood, whole blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, urine, stool, saliva, placental extracts, amniotic fluid, a cryoprecipitate, a cryosupernatant, a cell lysate, mammalian cell culture or culture medium, products of fermentation, ascitic fluid, proteins present in blood cells, solid tumours, for example, isolated from a mammal with a cancer, or any other specimen, or any extract thereof, obtained from a patient (human or animal), test subject, or experimental animal. A sample may also include, without limitation, products produced in cell culture by normal or transformed cells (e.g., via recombinant DNA or monoclonal antibody technology). A “sample” may also be a cell or cell line created under experimental conditions, that are not directly isolated from a subject. A sample can also be cell-free, artificially derived or synthesized.

### BRIEF DESCRIPTION OF THE DRAWINGS

FIGS. 1A-D show purification of the endonuclease from rat liver. A: Flow chart outlining a purification scheme, with the columns and fractionation listed on the left. Horizontal lines indicate an elution gradient. S indicates the supernatant and P is the precipitate. The fractions containing enzyme activity are in bold type. The final gradient resulting in the purification of the endonuclease is boxed at the bottom. B: 1000 units of endonuclease from each activity peak of each column except heparin-Sepharose were separated by SDS-PAGE and visualized by silver staining. Protein size markers (M) shown on the left are in kDa. C: Silver-stained SDS-PAGE from fractions 70-102 of heparin-Sepharose gradient eluted with increasing concentration of KCl ranging from 0.05 to 1.0 M. The candidate bands (˜34-35 and ˜10-25 kDa) corresponding to the endonuclease activity are indicated by arrows on the right. D: Autoradiograph for assaying the presence of endonuclease activity in post-heparin-Sepharose fractions (fractions 60-137) using 5′³²P-labeled CRD c-myc RNA as substrate. Fractions 70-102 correspond to the silver-stained SDS-PAGE in 1C. The control lane at 1D is partially purified post-Affigel enzyme. Unfilled arrow on the right indicates the undigested substrate RNA; filled arrow indicates endonucleolytic decay products generated by the endonuclease. Marker, indicated by numbers on the left, is 5′-labeled ³²P-pBR322 DNA cleaved with HaeII.

FIGS. 2A-B show an analysis of the endonuclease by glycerol gradient centrifugation and gel filtration. A: Enzyme from post-heparin Sepharose (0.2 ml) was centrifuged in a 10-70% glycerol gradient and aliquot from each fraction was assayed for endonuclease activity as described herein. Endonuclease activity was expressed as percent of maximal endonucleolytic product generated. A parallel gradient containing molecular mass marker proteins was spun, an aliquot from fractions was run on a 12% SDS-PAGE gel, and the location of protein standards were identified by silver staining. B: Gel filtration column was calibrated with molecular weight markers before chromatography with 1 mg of endonuclease under native conditions. Endonuclease activity was expressed as percent of maximal endonucleolytic product generated. Volumes at which protein standards were eluted as determined by silver-stained SDS-PAGE, and at which endonuclease activities were assayed, are shown on the y-axis.

FIGS. 3A-C show determination of in vitro cleavage sites on c-myc CRD RNA by the post-heparin Sepharose endonuclease. A: CRD RNAs corresponding to nts 1705-1792 (lanes 1-5) and 1705-1886 (lanes 6-9) were 5′³²P-end-labeled and subjected to endonuclease treatment (lanes 5 and 9) as described herein. These radiolabeled RNAs were also subjected to alkaline hydrolysis (lanes 2 and 6) and partial RNase T1 digests (lanes 3 and 7). Lanes 4 and 8 have no addition of enzyme under the standard endonuclease assay. Arrows on the left of autoradiogram indicate guanosine (G) cleavage sites generated by RNase T1 and the numbers indicate position of nucleotide sequences. Asterisks indicate the observable identical cleavage products generated by the endonuclease on both RNA substrates. B: Sequences on nt 1705-1812 of c-myc CRD RNA (SEQ ID NO: 1) correspond to the sites identified in (A). Underlined sequences and arrows indicate cleavage sites generated by the endonuclease. C: is a schematic diagram of human c-myc mRNA, indicating the location of the 1705-1792 coding region determinant (CRD).

FIG. 4 shows that the post-heparin Sepharose enzyme is an endonuclease. The 88-nt CRD RNA substrate (nts 1705-1792) was ³²P-labeled at its 5′ or its 3′-terminus. Each RNA was incubated under the standard endonuclease conditions for 5 min in the absence (lanes 3 and 8) or presence (lanes 4, 5, 9 and 10) of endonuclease. The reaction was then stopped and the RNA was harvested. Each RNA was then electrophoresed on a 12% polyarcylamide/7M urea gel, and the bands were visualized by phosphorImaging. Samples were run along side ³²P-labeled CRD RNAs which had been subjected to alkaline hydrolysis (lanes 1 and 6) or partial RNase T1 digest (lanes 2 and 7). Asterisks indicate the RNase T1 decay product (nt G1749) used as a reference point. Numbers 1-4 indicate the corresponding 5′ and 3′ ends endonucleolytic decay products generated using the 5′ and 3′ labeled RNAs.

FIG. 5 shows that the post-heparin Sepharose endonuclease is a protein-only enzyme. Three units of pre-treated endonuclease were incubated with 5′³²P-end-labeled CRD RNA for 5 min at 37° C. in the presence or absence of 5×10⁻² U of micrococcal nuclease (MNase) and 17 mM EGTA. The endonuclease pre-treatment consisted of incubating the enzyme with or without MNase at 37° C. for 5 min prior to addition of EGTA and 5′-labeled CRD RNA. Non-pre-treated endonuclease was used for lanes 2-8 and pre-treated enzymes were used for lanes 9 and 10. Lane 1 is 5′-end-labeled pBR322 ³²P-DNA cleaved with HaeII. Lane 2, no enzymes or EGTA; lane 3, endonuclease only; lane 4, endonuclease in the presence of EGTA; lane 5, MNase only; lane 6, MNase in the presence of EGTA; lane 7, endonuclease and MNase; lane 8, endonuclease, MNase and EGTA. Lane 9, endonuclease pre-treated with MNase and then with EGTA; lane 10, endonuclease pre-treated with water and then with EGTA. Arrow indicates the endonucleolytic decay products.

FIG. 6 shows that the post-heparin Sepharose endonuclease generates products with 3′ hydroxyls. 5′³²P-end-labeled CRD RNA was digested with 1 U of RNase T1 (lanes 3 and 4) for 1 min at 37° C., or with 2 U of S1 nuclease (lanes 6 and 7) or 3 U of the endonuclease (lanes 9 and 10) for 5 min at 37° C. The products of these reactions were then incubated for 10 min at 25° C. with 3×10⁻² U of snake venom exonuclease (lanes 4, 7 and 10), and electrophoresed on a 6% polyacrylamide/urea gel. The arrow indicates the position of the three characteristic products generated by the endonuclease. Lane 1 contains the 5′-end-labeled pBR322 ³²P-DNA HaeII-cleaved marker. Lanes 2, 5 and 8 are the starting substrate transcripts for each type of reactions.

FIGS. 7A-D show cleavage of various substrates by the post-heparin Sepharose endonuclease. A: CRD RNA and DNA corresponding to nts 1705-1792 of c-myc RNA were 5′³²P-end-labeled. Double-stranded (ds) nucleic acids were made as described herein. These different combinations of nucleic acids were run on a 10% non-denaturing gel, and bands containing nucleic acids of interest, shown by asterisks, were purified. B: Gel purified ³²P labeled single-stranded (ss) RNA, ss DNA, ds DNA, and DNA-RNA duplexes, as indicated, were subjected to the standard endonuclease assay as described herein. Samples were run on a 6% denaturing gel. Marker (M) in lane 1 is 5′-labeled ³²P-pBR322 DNA cleaved with HaeII. C: 5′³²P-end-labeled single- (lanes 4 and 5) and double-stranded (lanes 6 and 7) CRD RNAs corresponding to nts 1705-1886 of c-myc RNA were subjected to endonuclease assay, and samples were run on a 6% denaturing gel. Lanes 1 and 2 are single-stranded nts 1705-1792 CRD RNA. D: The same samples from lanes 4-7 in C were run on a 8% non-denaturing gel as indicated.

FIGS. 8A-C show various properties of the endonuclease. A: the endonuclease is insensitive to many known RNA inhibitors. B: the endonuclease has activity in the presence of various metal ions. C: the endonuclease is active at various temperatures.

FIG. 9 shows the effect of the post-heparin Sepharose endonuclease on various in-vitro transcribed RNAs. One unit of endonuclease was incubated for 5 min at 37° C. with the following deproteinized, 5′-end-labeled ³²P-RNAs: lane 1 and 2, 5′-CRD (c-myc nts 1705-1792); lanes 3 and 4, β-globin (nts 50-169); lanes 5 and 6, MDR1 (nts 745-922); and lanes 7 and 8, 5′ coding region of c-myc RNA (nts 1-270). Numbers on the left indicate sizes (nt) of the ³²P-RNA substrates.

FIG. 10 shows sites on the 1705-1792 CRD RNA sequences cleaved by the endoribonuclease. A: 12% denaturing polyacrylamide/urea gel showing eleven ribonuclease cleavage products (indicated on the right) as identified by comparison of partial digests of the CRD probe with RNase T1 and alkaline digestion. B: cleavage sites identified in A on the determined 1705-1792 CRD RNA (SEQ ID NO: 2) secondary structure (arrows).

FIG. 11 shows sites on mutant CRD RNA (1752 AU to UA) cleaved by the endoribonuclease. A: 12% denaturing polyacrylamide/urea gel showing the comparison between cleavage of the enzyme on both the wild-type and mutant CRD RNA. Results show that the enzyme is capable of cleaving both 1747UA and 1752UA. B: cleavage sites identified in A on the predicted secondary structure of mutant 1705-1792 CRD RNA (SEQ ID NO: 3) (arrows).

FIG. 12 shows a flow chart outlining an alternate purification scheme for the endonuclease from rat liver.

FIGS. 13A-B show analysis of endonuclease cleavage on SARS coronavirus RNAs. A: sequences of the constructed plasmids used to transcribe orf3 (SEQ ID NO: 4), spike (SEQ ID NO: 5) and orf1b (SEQ ID NO: 6) RNAs. B: 8% denaturing polyacrylamide/urea gel showing degradation of in-vitro transcribed orf3, spike, and orf1b RNAs.

FIG. 14 shows a 12% denaturing polyacrylamide/urea gel showing mapping of the cleavage sites generated by the endoribonuclease on in-vitro transcribed orf3, spike and orf1b RNAs.

### DETAILED DESCRIPTION OF THE INVENTION

The invention provides in part a novel endoribonuclease activity. The endoribonuclease can be purified as described herein, and is capable of preferentially cleaving RNA molecules at sites 3′ of uracil residues or sites 5′ of adenine residues.

Various alternative embodiments and examples of the invention are described herein. These embodiments and examples are illustrative and should not be construed as limiting the scope of the invention.

Endoribonuclease Compounds and Fusions

The endoribonuclease compounds of the invention may be derived from any sample or tissue, e.g., from human, rat, bovine, etc. The endoribonuclease compounds of the invention may be provided as fusion proteins by for example, covalently linking two protein segments. The covalent linkage may be reversible, such that the endoribonuclease is released upon delivery to a target cell or molecule. The endoribonuclease may be linked to another polypeptide using any means known in the art, for example, using chemical linkers such as a carbodiimide (e.g., 1-ethyl-3-(3-dimethylaminopropyl)carbodiimide (EDAC) or 1-ethyl-3-[3-dimethylaminopropyl]carboimide hydrochloride (EDC)), or other linker, such as, sulfosuccinimidyl-4-(N-maleimidomethyl)cyclohexane-1-carboxylate (Sulfo-SMCC) or N-hydroxysulfosuccinimide (Sulfo-NHS), etc.

Common proteins used for the second polypeptide include beta-galactosidase; beta-glucuronidase; green fluorescent protein (GFP); autofluorescent proteins, including blue fluorescent protein (BFP); glutathione-5-transferase (GST); luciferase, horseradish peroxidase (HRP); chloramphenicol acetyltransferase (CAT); maltose binding protein (MBP). Fusion proteins may also include epitope tags alternatively or additionally to the second polypeptide. Common epitope tags include histidine (His) tags, FLAG tags, influenza hemagglutinin (HA) tags, Myc tags, VSV-G tags, and thioredoxin (Trx) tags. Fusion proteins can also include Lex A DNA binding domain (DBD) fusions, GAL4 DNA binding domain fusions, and herpes simplex virus (HSV) BP16 protein fusions. Fusion proteins can also include a targeting moiety, for example, an antibody that directs the fusion protein to specific cells, such as cancer cells, viruses, or cells infected by a virus.

Test Compounds

Test compounds may be identified from large libraries of both natural products or synthetic (or semi-synthetic) extracts or chemical libraries according to methods known in the art. Those skilled in the field of drug discovery and development will understand that the precise source of test extracts or compounds is not critical to the method(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla., USA), and PharmaMar, MA, USA. In addition, natural and synthetically produced libraries of compounds are produced, if desired, according to methods known in the art, e.g., by standard extraction and fractionation methods. Furthermore, if desired, any library or compound is readily modified using standard chemical, physical, or biochemical methods.

When a crude extract is found to modulate endoribonuclease activity, further fractionation of the positive lead extract is necessary to isolate chemical constituents responsible for the observed effect. Thus, the goal of the extraction, fractionation, and purification process is the careful characterization and identification of a chemical entity within the crude extract having endoribonuclease modulatory activities. The same assays described herein for the detection of activities in mixtures of compounds can be used to purify the active component and to test derivatives thereof. Methods of fractionation and purification of such heterogeneous extracts are known in the art. If desired, compounds shown to be useful agents for treatment are chemically modified according to methods known in the art. Compounds identified as being of therapeutic, prophylactic, diagnostic, or other value may be subsequently analyzed using a suitable animal model for cell proliferation, viral infection, or aberrant RNA expression disorders.

Laboratory or Clinical Applications

The endoribonuclease compounds of the invention may be used as laboratory or clinical agents in a variety of research or clinical applications or assays.

In some embodiments the endoribonuclease compounds may be used in a mode similar to DNA restriction enzymes (DNA endonucleases) to cleave RNA at, for example, phosphodiester bonds 3′ of U and 5′ of A of non-base paired sites or at other sites as described herein.

In some embodiments, the endoribonuclease compounds may be used to eliminate RNA (e.g., mRNA) present in a cell or sample to, for example, isolate DNA present in the cell or sample, or to disrupt protein expression within a cell. This may be done by for example exposing the cell or sample to the endoribonuclease compounds under conditions suitable for degrading RNA molecules, for example under the endonuclease assay conditions described herein or known in the art.

In some embodiments, the endoribonuclease compounds may be used to study the physical secondary structure of a specific RNA molecule (e.g. a RNA molecule that is in solution) by taking advantage of the ability of the endoribonuclease compounds to preferentially cleave non-base paired RNA.

In some embodiments, any RNA, including mRNA and structural RNAs, can be targeted for degradation using the endoribonuclease compounds of the invention. For example, introduction of a complementary antisense RNA sequence corresponding to a specific site of a target mRNA (e.g., a RNA probe), under conditions that will facilitate hybridization of the RNA molecule with the mRNA of the gene, in combination with introduction of an endoribonuclease compound of the invention, results in degradation of the resulting double-stranded RNA. Elimination of the RNA of a specific gene may be useful for example to study the function of the gene. In alternative embodiments, introduction of an endoribonuclease compound of the invention into a target cell results in the degradation of the RNA within the cell.

In some embodiments, the endoribonuclease compounds may be used to assay the presence or absence of specific cleavage sites or to map the location of sequences on a RNA molecule for example by annealing the RNA molecule with a complementary RNA probe and cleaving the resulting double stranded RNA with the compound.

In some embodiments, the endoribonuclease compounds may be used to detect mutations or mismatches by annealing a RNA molecule with a complementary DNA probe, where a fully complementary RNA/DNA duplex would not be sensitive to cleavage by the endoribonuclease compound, while a mutation or mismatch that created single stranded, non-complementary portions would be cleaved by the endoribonuclease compound.

In some embodiments, the endoribonuclease compounds may be used to isolate specific DNA sequences or molecules from a cell or sample by annealing the DNA molecule with a complementary RNA probe, where the RNA/DNA duplex would not be sensitive to cleavage by the endoribonuclease compound, but non-hybridized RNA could be eliminated due to single stranded endoribonuclease activity. If desired, the RNA probe could be degraded by for example RNase H treatment after isolation of the duplex to leave intact the DNA molecule of interest.

In some embodiments, the endoribonuclease compounds of the invention may be used to identify endoribonucleases having similar chemical or physical properties, or to identify agents capable of modulating the endoribonuclease activity, by for example comparing the chemical or physical properties of the test molecule with the endoribonuclease compounds of the invention.

The various applications may be carried out under conditions suitable for optimal activity of the compounds of the invention. For example, endoribonuclease reactions may be carried out at a temperature of at least 45° C. to about 65° C. for at least 10 minutes. In other embodiments, endoribonuclease reactions may be carried out at a temperature of about 4° C. In other embodiments, endoribonuclease reactions may be carried out in the presence of inhibitors known to inactivate other RNases. In other embodiments, endoribonuclease reactions may be carried out in the presence of metal ions such as magnesium and calcium ions. The reactions may be inactivated for example by adding an excess of copper ions. The reactions may be carried out in cell-free conditions, or may be carried out using cells obtained from various sources as described herein or known to those of skill in the art.

Assays

Various assays, as described herein or known to one of ordinary skill in the art, may be performed to determine the modulatory activity of a test compound or to eliminate, target, or protect RNA molecules as described herein or as known by one of ordinary skill in the art.

Endoribonuclease compounds may be provided in cells or cell lysates from, for example, animal tissue (e.g., rat liver, bovine liver, etc). Cells and cell lines may be obtained from commercial sources, for example, ATCC, Manassas, Va., USA. Cells and tissues may also be derived from subjects having any of the disorders described herein. For example, cell lines used as models of proliferative diseases may include commercially available cells from, for example, the American Type Culture Collection (ATCC), Manassus, Va., USA. Such cell lines may include LnCaP cells, HeLa cells, Daudi cells, Raji cells, HEK 293 cells, K562, MCF-7, HL-60, etc. Suitable animal models, e.g., of proliferative diseases include, for example, transgenic rodents (e.g. mice, rats) bearing gain of function proto-oncogenes (e.g. Myc, Src) and/or loss of function of tumour suppressor proteins (e.g. p53, Rb) or rodents that have been exposed to radiation or chemical mutagens that induce DNA changes that facilitate neoplastic transformation. Many such animal models are commercially available, for example, from The Jackson Laboratory, ME, USA. These animal models may be used as source cells or tissue for the assays of the invention. Test compounds may also be assayed in these models.

The assays may be conducted using detectably labelled molecules, i.e., any means for marking and identifying the presence of a molecule, e.g., an oligonucleotide probe or primer, a gene or fragment thereof, a peptide, or a cDNA molecule. Methods for detectably-labelling a molecule are well known in the art and include, without limitation, radioactive labelling (e.g., with an isotope such as ³²P or ³⁵S) and nonradioactive labelling such as, enzymatic labelling (for example, using horseradish peroxidase or alkaline phosphatase), chemiluminescent labeling, fluorescent labeling (for example, using fluorescein), bioluminescent labeling, or antibody detection of a ligand attached to the probe. Also included in this definition is a molecule that is detectably labelled by an indirect means, for example, a molecule that is bound with a first moiety (such as biotin) that is, in turn, bound to a second moiety that may be observed or assayed (such as fluorescein-labeled streptavidin). Labels also include digoxigenin, luciferases, and aequorin.

Therapeutics and Prophylactics

Endoribonuclease compounds according to the invention may be used for treatment or prophylaxis of disorders that would benefit from a reduction in the quantity of RNA e.g., disorders associated with the aberrant expression or overexpression of RNA or by excessive or aberrant cell proliferation that may include RNA overexpression as a component. Such disorders may include prion diseases (e.g., Creuzfeldt-Jakob Disease (CJD) and vCJD in humans, or bovine spongiform encephalopathy (BSE) in cows) or disorders associated with expression of microRNAs or RNAs transcribed from introns or antisense DNA. Cell proliferative diseases and disorders include, for example, neoplasms, such as fibrosarcoma, myosarcoma, liposarcoma, chondrosarcoma, osteogenic sarcoma, chordoma, angiosarcoma, endotheliosarcoma, lymphangiosarcoma, lymphangioandotheliosarcoma, synoviome, mesothelioma, Ewing's tumor, leiomyosarcoma, rhabdomyosarcoma, gastric cancer, esophageal cancer, colon carcinoma, rectal cancer, pancreatic cancer, breast cancer, ovarian cancer, prostate cancer, uterine cancer, cancer of the head and neck, skin cancer, brain cancer, squamous cell carcinoma, sebaceous gland carcinoma, papillary carcinoma, papillary adenocarcinoma, cystadenocarcinome, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular cancer, lung carcinoma, small call lung carcinoma, non-small cell lung carcinoma, bladder carcinoma, epithelial carcinoma, glioma, astracytoma, medulloblastoma, craniopharyngioma, ependymoma, pinealoma, hemangloblastoma, acoustic neuroma, oligodendroglioma, meningioma, melanoma, neuroblastoma, retinoblastoma, leukemia, lymphoma, mantle cell lymphoma or Kaposi's sarcoma. Disorders of cell proliferation also include haematopoietic diseases, psoriasis, atherosclerosis, dermatological diseases, such as pemphigus vulgaris and pemphigus foleaceus, inflammatory disorders, e.g., Crohn's disease, rheumatoid arthritis, ulcerative colitis, organ transplants.

In some embodiments, endoribonuclease compounds according to the invention are capable of cleaving and degrading c-myc RNA. C-myc is a transcription factor that plays a fundamental role in controlling cell growth, differentiation, and death in virtually all mammalian cells, which is consistent with its ability to bind to several thousand coding sequences. Aberrant c-myc activity, e.g., amplification or overexpression, at both polypeptide and nucleic acid levels, has been implicated in the pathogenesis of cell proliferative disorders such as various cancers (Dang C V, Mol. Cell Biol. 19:1-11, 1999), including bladder cancer, breast cancer, colon cancer, gastric cancer, hepatocellular cancer, leukemia, lymphoma, glioblastoma, cervical cancer, melanoma, neuroblastoma, ovarian cancer, prostate cancer, rhabdomyosarcoma, small-cell lung cancer, uveal melanoma, etc. C-myc has also been implicated in the pathogenesis of restenosis (Lee et al., Antisense and Nucleic Acid Dev 9:487-492, 1999).

By a “cancer” or “neoplasm” is meant any unwanted growth of cells serving no physiological function. In general, a cell of a neoplasm has been released from its normal cell division control, i.e., a cell whose growth is not regulated by the ordinary biochemical and physical influences in the cellular environment. In most cases, a neoplastic cell proliferates to form a clone of cells which are either benign or malignant. Examples of cancers or neoplasms include, without limitation, transformed and immortalized cells, tumours, and carcinomas such as breast cell carcinomas and prostate carcinomas. The term cancer includes cell growths that are technically benign but which carry the risk of becoming malignant. By “malignancy” is meant an abnormal growth of any cell type or tissue. The term malignancy includes cell growths that are technically benign but which carry the risk of becoming malignant. This term also includes any cancer, carcinoma, neoplasm, neoplasia, or tumor

Endoribonuclease compounds according to the invention may be used for treatment or prophylaxis of disorders related to viral infection, for example, by SARS virus, human immunodeficiency virus (HIV), Human T-cell Lymphotrophic virus (HTLV), human papillomavirus (HPV), Hepatitis A, B, or C virus, herpesviruses (e.g., herpes simplex virus, varicella herpes zoster virus, cytomegalovirus, or Epstein Barr virus), paramyxoviruses, polioviruses, rhinoviruses, adenoviruses, coronaviruses, or viruses that cause rubella, measles, mumps, rabies, ebola, or influenza. In some embodiments, endoribonuclease compounds according to the invention are capable of cleaving and degrading SARS virus RNAs, such as orf3, orf1b, or spike RNA. Endoribonuclease compounds according to the invention may also be used for treatment or prophylaxis of disorders related to infection, for example by a bacterium, yeast, parasite, helminth, etc.

Pharmaceutical & Veterinary Compositions, Administration, and Dosages

Endoribonuclease compounds of the invention can be provided alone or in combination with other compounds (for example, nucleic acid molecules, small molecules, peptides, or peptide analogues), in the presence of any pharmaceutically acceptable carrier, in a form suitable for administration to animals such as mammals, for example, humans, cattle, sheep, etc. If desired, treatment with an endoribonuclease compound according to the invention may be combined with more traditional and existing therapies for cell proliferative disorders, viral infections, or aberrant RNA expression disorders.

Conventional pharmaceutical practice may be employed to provide suitable formulations or compositions to administer the endoribonuclease compounds to subjects suffering from or presymptomatic for cell proliferative disorders, viral infection, or RNA expression disorders. Methods well known in the art for making formulations are found in, for example, “Remington's Pharmaceutical Sciences” (19^{th} edition), ed. A. Gennaro, 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic compositions, the compounds are administered to an individual in an amount sufficient to stop or slow a cell proliferative disorder, a viral infection, or a RNA expression disorder, depending on the disorder.

The compounds according to the invention may be administered in combination (e.g, by covalent or non-covalent binding, as a fusion) with a targeting molecule, e.g., an antibody that specifically recognizes a cell, such as a cell that is undergoing inappropriate cell proliferation or RNA expression, or that is infected with for example a virus or other infectious organism, for delivery to that cell. For example, malignant B cells overexpress CD22 and an antibody to CD22 would therefore target B cells. Such targeting methods are known in the art, and are described in for example U.S. Pat. No. 5,541,297 issued to Hansen et al. Jul. 30, 1996; U.S. Pat. No. 4,867,973 issued to Goers et al. Sep. 19, 1989; or U.S. Pat. No. 5,776,427 issued to Thorpe et al. Jul. 7, 1998. Targeting molecules may also include polypeptides such as the antennapedia transducing protein (Chikh G G et al. (2001) J. Immunology 167: 6462-6470), that is capable of delivering a fusion polypeptide into a cell.

The endoribonuclease compounds according to the invention may be administered in combination, in a single formulation or as separate formulations, with for example an antisense RNA molecule that is complementary to a gene of interest such that the compound may specifically degrade the double stranded molecules formed as a result of hybridization between the mRNA of the gene of interest and the complementary RNA molecule.

Any appropriate route of administration may be employed, for example, parenteral, intravenous, subcutaneous, intramuscular, intracranial, intraorbital, ophthalmic, intraventricular, intracapsular, intraspinal, intrathecal, intracisternal, intraperitoneal, intranasal, aerosol, topical, or oral administration. Formulations may be in the form of liquid solutions or suspensions; for oral administration, formulations may be in the form of tablets or capsules; and for intranasal formulations, in the form of powders, nasal drops, or aerosols. For treatment of restenosis, for example, the therapeutic or prophylactic formulations may be coated on a stent. The endoribonuclease compounds may be provided in liposomes.

An “effective amount” of an endoribonuclease compound according to the invention includes a therapeutically effective amount or a prophylactically effective amount. A “therapeutically effective amount” refers to an amount effective, at dosages and for periods of time necessary, to achieve the desired therapeutic result, such as reduction of cell proliferation, viral infection, or aberrant RNA expression, or reduction of diseased cells or tissue. A therapeutically effective amount of an endoribonuclease compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the endoribonuclease compound are outweighed by the therapeutically beneficial effects. A “prophylactically effective amount” refers to an amount effective, at dosages and for periods of time necessary, to achieve the desired prophylactic result, such as reduction of cell proliferation, viral infection, or aberrant RNA expression, or reduction of diseased cells or tissue. Typically, a prophylactic dose is used in subjects prior to or at an earlier stage of disease, so that a prophylactically effective amount may be less than a therapeutically effective amount. An exemplary range for therapeutically or prophylactically effective amounts of an endoribonuclease compound may be any number from 0.1 nM-0.1M, 0.1 nM-0.05M, 0.05 nM-15 μM or 0.01 nM-10 μM.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In the case of vaccine formulations, an immunogenically effective amount of an endoribonuclease compound of the invention can be provided, alone or in combination with other compounds, with an immunological adjuvant, for example, Freund's incomplete adjuvant, dimethyldioctadecylammonium hydroxide, or aluminum hydroxide. The compound may also be linked with a carrier molecule, such as bovine serum albumin or keyhole limpet hemocyanin to enhance immunogenicity.

In general, endoribonuclease compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

### EXAMPLE 1

Purification of a novel mammalian ribonuclease activity was undertaken essentially as described in Lee et al. (J. Biol. Chem. 273:25261-71, 1998) with some modifications. The modifications include: (i) addition of the pH precipitation step, (ii) omission of the Q-Sepharose step, and (iii) addition of the Affi-gel step, as described in greater detail below.

Materials

Frozen livers from male Sprague-Dawley rats purchased from Harlan Bioproducts (Madison, Wis.) were used for preparation of ribosomal salt wash and partially purified proteins described herein. T4 polynucleotide kinase and ribonucleoside vanadyl complex were from New England Biolabs (Ontario. Canada). Alkaline phosphatase and RNase T1 were obtained from Roche Applied Sciences (Laval, Quebec). SP6 Megascript kits were purchased from Ambion (Austin, Tex.). RNasin was purchased from Promega (Madison, Wis.) and [γ-³²P] ATP (3000 Ci/mmol) was from Amersham Biosciences (Montreal, Quebec). All other chemicals were either purchased from VWR or Fisher Scientific (Ontario, Canada).

Preparation of Ribosomal Salt Wash

Frozen livers from male Sprague-Dawley rats were used for all protein purification experiments described herein. All procedures were performed on ice unless otherwise indicated. Frozen livers were homogenized in Buffer A (1 mM potassium acetate, 1.5 mM magnesium acetate, 2 mM DTT, 10% (v/v) glycerol, 0.1 mM EGTA, 10 mM Tris-Cl, pH 7.4) (1 ml Buffer A for every 0.4 g tissues) for 2-3 mins using a Polytron. Subsequent steps for the preparation of liver polysomes and salt-eluted polysomal proteins designated as ribosomal salt wash (RSW) have been previously described (Lee et al., 1998, supra). Typically, between 50 and 75 mg of RSW proteins can be obtained from one liver weighing 175-200 g.

Preparation of Radiolabeled Nucleic Acids

The plasmid DNA, pUC19-CRDmyc, used herein was constructed by sub-cloning a PCR product corresponding to nucleotides 1705-1792 of human c-myc mRNA into BamHI and EcoRI restriction sites of pUC19. The PCR product was generated using primers FUP (5′-CTCGGATCCATTTAGGTGACACTATAGACCAGATCCCGGAGTTGG (SEQ ID NO: 7), corresponding to nts 1705-1722 of human c-myc mRNA and containing a BamHI restriction site as indicated by underlined nucleotides) and RUP (5′-CTCGAATTCGCTTGGACGGACAGGATG (SEQ ID NO: 8), corresponding to nt 1775-1792 of human c-myc mRNA and contained a EcoRI restriction site as indicated by underlined nucleotides), using the human c-myc cDNA as template. The RNA corresponding to c-myc nts 1705-1792 is referred to herein as CRD RNA. The 5′-³²P-end labeling of CRD RNA was used as the substrate for purifying and characterizing liver endonucleases. Unlabeled CRD RNA was synthesized by linearizing pUC19-CRDmyc with EcoRI and transcribing the DNA with SP6 polymerase using SP6 Megascript kits (Ambion, Tex.). The plasmid DNA, pGEM4Z-myc 705-1886 was made by sub-cloning a PCR product corresponding to nucleotides 1705-1886 of human c-myc mRNA into BamHI and HindIII restriction sites of pGEM4Z. The PCR product was generated using primers F1705-1722 (5′CTCGGATCCACCAGATCCCGGAGTTGG, SEQ ID NO: 9) and R1869-1886 (5′CGACAAGCTTCGCACAAGAGTTCCGTAG, SEQ ID NO: 10), using human c-myc cDNA as template. Unlabeled sense RNA corresponding to nts 1705-1886 was made by linearizing the DNA with HindIII and transcribing the DNA with SP6 polymerase. To make anti-sense RNA corresponding to nts 1705-1886, the DNA was linearized with EcoRI and transcribed with T7 RNA polymerase.

For 5′-³²P-end labeling of RNA, 5 μg of in-vitro transcribed RNA was first dephosphorylated with 10 units of alkaline phosphatase for 30 min at 37° C. in a 100-μl reaction according to the manufacturer's instructions. Dephosphorylated RNA was purified by phenol/chloroform extraction and ethanol precipitation. 2.5 μg of dephosphorylated RNA was incubated in a 25-μl reaction with 30-50 μCi of [γ-³²P] ATP at 37° C. for 1 h with 40-50 units of T4 polynucleotide kinase. The entire sample was then run on a 6% polyacrylamide/7M urea gel and the band containing 5′-labeled ³²P-RNA was sliced out and eluted with elution buffer (0.5 M ammonium acetate, 1 mM EDTA, 0.2% SDS) at 37° C. for 6 h. The purified radiolabeled RNA was then concentrated by ethanol precipitation.

Oligonucleotides myc1705-1792 and myc1792-1705 were purchased from QIAGEN (Alameda, Calif.). 5′-³²P-end labeling of DNA was essentially as described above for 5′-end labeling for RNA.

For preparation of double-stranded RNA or DNA, 1 ng of 5′-labeled ³²P-RNA or DNA was first heated to 80°-90° C. for 5 mins with 5 ng of complementary DNA or RNA strand in 40 mM PIPES, pH 6.4, 400 mM NaCl, 1 mM EDTA and 80% formamide. The nucleic acids were then allowed to hybridize overnight at 52° C., followed by ethanol precipitation. The double-stranded RNA, DNA or RNA-DNA duplexes were then purified from a 10% non-denaturing polyacrylamide gel.

In Vitro Assay for Endonuclease Activity

The pH of all buffers for experiments described here was determined at room temperature. The standard 20-25-μl reaction mixture used for this assay included 2 mM DTT, 1.0 unit of RNasin, 2 mM magnesium acetate, 50 mM potassium acetate, 0.1 mM spermidine, 1 ng of 5′-end-labeled ³²P-RNA (approximately 5×10⁴ cpm), and 10 mM Tris-HCl, pH 7.4. Reactions were incubated for 5 min at 37° C. unless otherwise indicated, placed in liquid nitrogen, and then at 80-90° C. to inactivate enzyme activities. Five μl of loading dye (9M urea, 0.2% xylene cyanol, 0.2% bromophenol blue) were added to 5 μl of reaction samples, and then subjected to electrophoresis for 75 mins in a 6% polyacrylamide, 7 M urea gel. Gels were fixed in 10% acetic acid, 10% methanol for 15 min, and then dried and exposed to a PhosphorImager screen (Cyclone PhosphorImager).

Purification of Liver Polysomal Endoribonuclease

The pH of approximately 28 g of RSW proteins was adjusted to 5.0 using HCl, and then spun at 18,000×g for 20 min at 4° C. to pellet precipitated proteins. The supernatant was recovered and the pH was adjusted to 7.0 using sodium hydroxide. Approximately 1.8 g of pH 7.0-adjusted RSW proteins were loaded at a flow rate of 1.8 ml/min on 5×48 cm phosphocellulose column (Sigma) equilibrated with 0.05 M KCl in 25 mM potassium phosphate, pH 6.0 (Buffer B). The column was washed with three volumes of Buffer B, or until the absorbance at 280 nm (A280) had returned to baseline. The column wash was collected and stored at 4° C. Bound proteins were eluted with a linear gradient from 0.05 to 1.0 M KCl in three volumes of Buffer B. 30 ml fractions were collected and 1 μl aliquots were taken from every fourth fraction for endonuclease activity assay as described herein. Weak endonuclease activity was detected in the column wash. Therefore, any column wash containing endonuclease activity was reloaded directly onto the phosphocellulose column in a separate run. Twelve separate phosphocellulose columns were conducted, and each column yielded highly reproducible results. Fractions containing at least 60% of maximal endonuclease activity from each column were pooled.

The pooled phosphocellulose fractions were diluted to approximately 0.1 M KCl by the addition of three volumes of 0.05 M KCl in 50 mM triethanolamine, pH 7.4 (Buffer C). It was then loaded at a flow rate of 1 ml/min onto 2.5×16 cm Reactive blue-4 column (Sigma) equilibrated with Buffer C. The column was washed with five volumes of Buffer C, or until A₂₈₀ had returned to zero. Bound proteins were eluted with a linear gradient from 0.05 to 1.0 M KCl in five volumes of Buffer C. 3.0 ml fractions were collected and assayed for presence of endonuclease activity, and active fractions containing 60% of maximal endonuclease activity were pooled. Fourteen separate 2.5×16 cm Reactive blue 4 columns were conducted, and each produced similar results.

To dilute the pooled Reactive blue-4 sample to 0.1 M KCl, four volumes of Buffer C were added. It was then loaded at a flow rate of 1 ml/min onto a 2.5×16 cm Reactive green-19 column (Sigma) and equilibrated with Buffer C. The column was washed with Buffer C, and bound proteins were eluted with a linear gradient from 0.05 M KCl to 1.0 M KCl in five volumes of the same buffer. 2.0 ml fractions collected were assayed for endonuclease activity. Two separate Reactive green-19 columns were conducted, and each yielded similar results. Fractions containing at least 60% of maximal endonuclease activity were pooled.

To dilute the pooled Reactive green-19 samples to approximately 0.1 M KCl, seven volumes of Buffer C were added. It was then loaded at a flow rate of 0.75 ml/min onto a 2.5×14 cm Affi-Gel Heparin gel column (Bio-Rad) and equilibrated with Buffer C. The column was washed with 5 volumes of Buffer C, and bound proteins were eluted with a linear gradient from 0.05 to 1.0 M KCl in five volumes of the same buffer. 1.5 ml fractions were collected and assayed for endonuclease activity. Fractions containing at least 70% of maximal endonuclease activity were pooled. Two separate Affi-Gel Heparin gel columns were conducted, and each produced similar results.

To dilute the pooled Affi-Gel Heparin gel fractions to approximately 0.1 M KCl, five volumes of Buffer C were added. The diluted pooled fractions were applied at a flow-rate of 0.75 ml/min onto a 5.0 ml pre-packed heparin-Sepharose column (Amersham Bioscience, Quebec) and equilibrated with Buffer C. The column was washed with 10 volumes of Buffer C, and bound proteins were eluted with a linear gradient from 0.05 to 1.0 M KCl in five volumes of the same buffer. 0.25 ml fractions collected were assayed immediately for endonuclease activity. Fractions containing at least 70% of maximal endonuclease activity were pooled. Two separate heparin-Sepharose columns were performed, and each yielded similar results.

One unit of post-heparin Sepharose endonuclease was used for most experiments described throughout this study unless otherwise indicated, and was defined as the amount of enzyme required to cleave ˜30% of the input RNA substrate into decay product in a 5-min reaction at 37° C.

Glycerol Gradient Centrifugation of Purified Enzyme

Purified enzyme (0.2 ml of post-heparin-sepharose sample) was layered onto a 10-70% (v/v) glycerol gradient (3.85 ml, 11×60-mm tubes) made in 0.25 M KCl, 0.1 mM EDTA, 50 mM Tris-HCl, pH 7.4, and was centrifuged for 18 h, 4° C., 200,000×g (44,100 rpm) in a Beckman SW 60 rotor. Fractions of 100 μl (for protein standards) or 200 μl (for RSW and post-heparin) were collected manually from the top. 4 μl of each fraction (for RSW and post-heparin) were assayed for endonuclease activity. A separate 10-70% glycerol gradient was centrifuged in the same manner and contained the following proteins as molecular mass standards (Amersham Biosciences, Quebec): myosin (205 kDa), β-galactosidase (116 kDa), phosphorylase b (97 kDa), transferrin (80 kDa), albumin (66 kDa), glutamate dehydrogenase (55 kDa), ovalbumin (45 kDa), carbonic anhydrase (30 kDa), trypsin inhibitor (21 kDa), lysozyme (14 kDa), and aprotinin (6.5 kDa). The protein standards were identified by silver staining of SDS-PAGE.

Gel Filtration Chromatography of Enzyme

Enzyme from post-phosphocellulose column (3 ml; 1 mg of protein) was first dialyzed overnight in 0.15 M KCl, 50 mM triethanolamine, pH 7.4 at 4° C. using PIERCE Slide-A-Lyzer dialysis cassette. Dialyzed enzyme was then chromatographed at room temperature on a Sephacryl S-100 (Amersham) column equilibrated with 0.15 M KCl, 50 mM triethanolamine, pH 7.4. The column was run at 0.5 ml/min, and 0.4-ml fractions were collected. A 4-μl aliquot of fractions was assayed for endonuclease activity. The column was calibrated with myoglobin (17 kDa), ovalbumin (45 kDa), and SIGMA molecular weight markers: Blue dextran 2000 (2,000 kDa), β-amylase (200 kDa), alcohol dehydrogenase (150 kDa), bovine serum albumin (66 kDa), carbonic anhydrase (29 kDa), cytochrome c (12.4 kDa). The elution of these proteins was determined by absorbance at 280 nm and further confirmed by silver stained SDS-PAGE.

Purification of the Liver Endonuclease

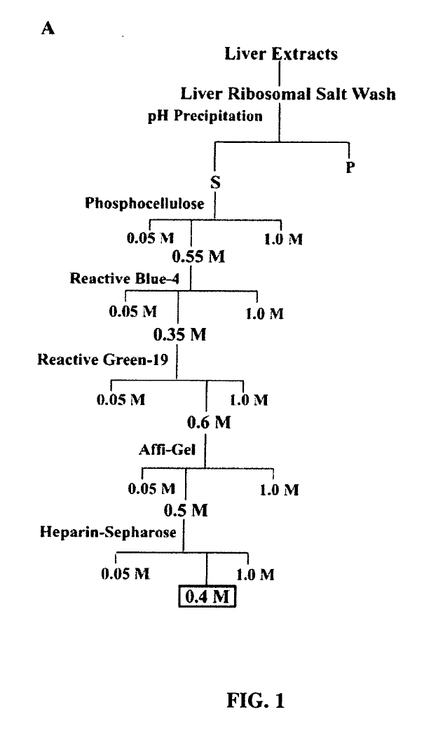

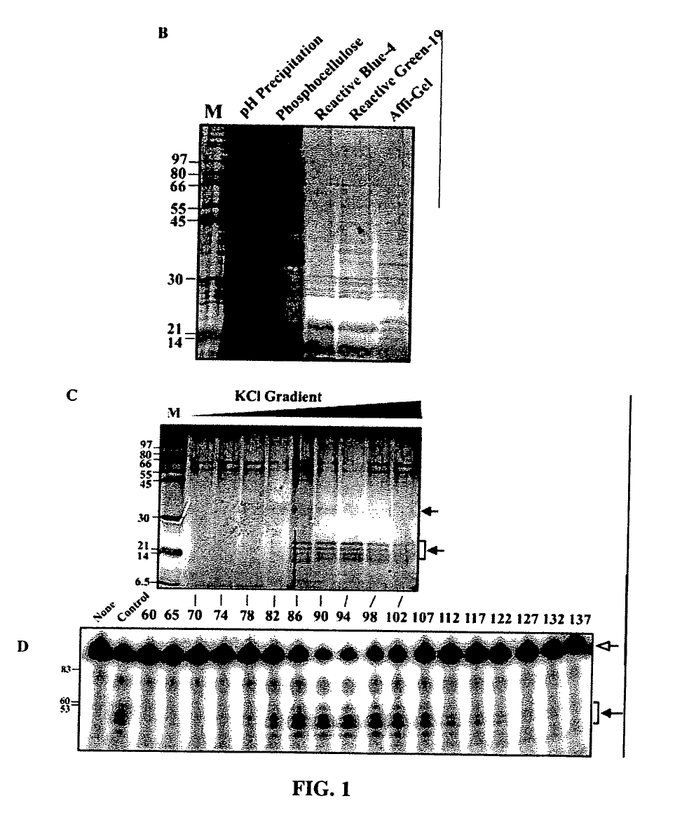

The first step in the purification was the isolation of proteins in high salt ribosomal salt washes (RSW) from rat liver. This step was achieved by the removal of nuclei and the bulk of cytoplasmic organelles, thus generating crude polysomal extracts. Proteins that were eluted with high salt from the crude polysomes designated as RSW were used as the starting material. The enzyme was purified through five column chromatographic steps and one non-chromatographic approach. The purification scheme is outlined in FIG. 1A. We first subjected the RSW to the non-chromatographic pH-precipitation step, as it increased the purification by approximately 4-fold (Table I).

TABLE I
Purification of Rat Liver Endonuclease

| | Total | | | Specific | | |
|---|---|---|---|---|---|---|
| | protein | Volume | Activity | activity | | Purification |
| Step | mg | ml | Units | Units/mg | Yield % | Fold |
| Ribosomal salt wash | 28,922 | 8,799 | 3.2 × 10⁷ | 1,108 | 100 | 1 |
| PH precipitation | 20,364 | 8,799 | 9.7 × 10⁷ | 4,753 | >100 | 4.3 |
| Phosphocellulose | 667 | 7,133 | 1.6 × 10⁷ | 23,733 | 49 | 21 |
| Reactive blue-4 | 3.7 | 491 | 1.1 × 10⁶ | 287,719 | 3.3 | 260 |
| Reactive green-19 | 0.36 | 102 | 2.6 × 10⁵ | 710,753 | 0.8 | 642 |
| Affigel | 0.11 | 46 | 9.4 × 10⁴ | 926,764 | 0.3 | 837 |

Endonuclease activity was determined in the linear range at each step of the purification except after the heparin-Sepharose column. One unit of endonuclease was defined as the amount of enzyme required to cleave 30% of the input RNA substrate into decay products in a 5-min reaction at 37° C.

Following the adjustment of pH back to 7, the proteins in the supernatant were bound to phosphocellulose and eluted with an increasing KCl gradient up to 1.0 M KCl. Proteins in active fractions eluted at ˜0.55 M KCl were pooled, diluted and bound to a reactive blue-4 column. Proteins in active fractions eluted at ˜0.35 M KCl from reactive blue-4 column were pooled, diluted and bound to reactive green-19 column. Proteins that eluted at ˜0.6 M KCl which contained endonuclease activity, were pooled, diluted and bound to an Affi-gel column. Active fractions eluted at ˜0.5 M KCl from the Affi-gel column were also pooled and diluted. Specific activity of the endonuclease was measured at each purification step as shown in Table I. The table also shows that the endonuclease can be purified up to about 840-fold on the fourth chromatographic step, the Affi-gel column. The degree of purification fold after the final chromatography step, the heparin-Sepharose column, could not be determined because the amount of protein recovered was too small. This information is therefore not included in Table I. Pooled active fractions from each purification step, from pH precipitation to Affi-gel column, were resolved on SDS-PAGE and visualized by silver staining as shown in FIG. 1B.

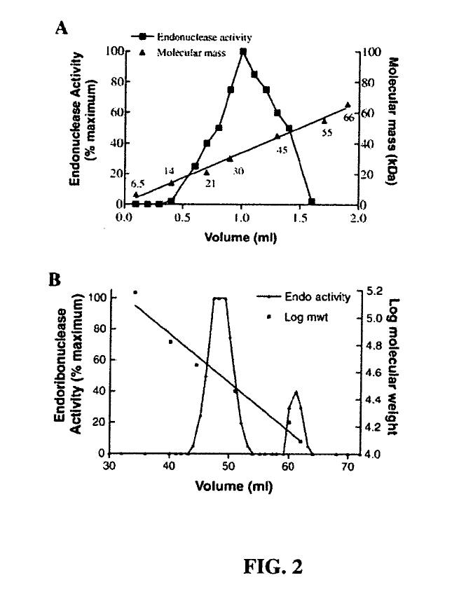

Proteins from pooled and diluted active fractions from Affi-gel were bound to the last purification column heparin-sepharose, and eluted with an increasing KCl gradient from 0.05 to 1.0 M KCl. Aliquots from the active and surrounding fractions from heparin-sepharose column were resolved on a SDS-PAGE and visualized by silver staining shown in FIG. 1C. The assay for endonucleolytic activity for the corresponding fractions is shown at FIG. 1D. The endonuclease activity recovered from heparin-sepharose column contained a few observable proteins (see fraction 86 of FIG. 1C). A comparison of enzyme activity, as indicated by intensity of degradation products (filled arrow, FIG. 1D), with the protein elution profile of fractions 82-102 (FIG. 1C) showed that there are five bands, one at ˜34-35 kDa (shown by arrow) and four at ˜10-25 kDa (shown by arrow) on SDS-PAGE. The 34-35 kDa band corresponded to the endonuclease activity. To determine the sizes of proteins which contribute to the endonuclease activity, glycerol gradient centrifugation and gel filtration experiments were performed. A glycerol gradient centrifugation experiment using the enzyme shown in FIG. 2A, indicated that the endonuclease activity peaks at ˜1.0 ml from top of the tube. This corresponded with the sedimentation of protein at ˜34-35 kDa. Gel filtration chromatography was performed under non-denaturing conditions. As shown in FIG. 2B, two endonuclease activities were visible, one significantly more prominent at elution volume of ˜48 ml which corresponded to ˜34-35 kDa protein, and the other much weaker activity eluted at volume of ˜61.2 ml which corresponded to ˜13 kDa protein. The endonuclease had a molecular weight of about 34-35 kDa, was stable and could be purified without the addition of a carrier protein.

CRD Cleavage Sites Generated by the Endonuclease

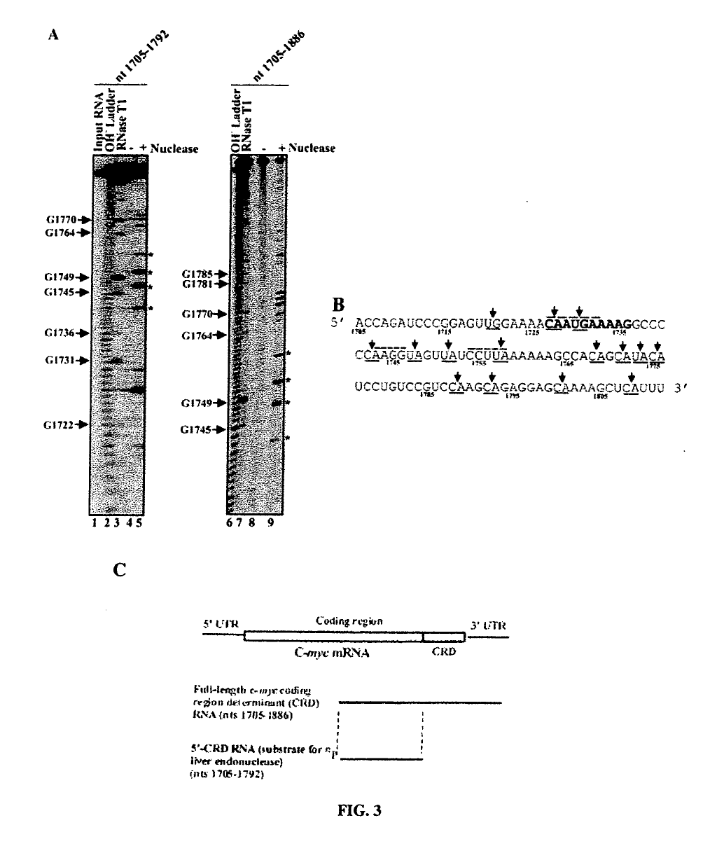

To map the cleavage sites generated by the endonuclease, a probe corresponding to the coding region determinant (CRD) of human c-myc mRNA (FIG. 3C) was used. We ran c-myc 5′-CRD ³²P-RNA substrates that had been treated with the enzyme along side with c-myc 5′-CRD ³²P-RNAs that had been subjected to a partial RNase T1 digestion and alkaline hydrolysis (FIG. 3A). Under partial digestion conditions, RNase T1 preferentially cleaves after single-stranded guanosine (G) residues in regions that are accessible. Fragments generated by partial RNase T1 digest, as shown by left hand pointed arrows in FIG. 3A, were successfully identified, and G sites were mapped accordingly. Using these mapped G sites and the alkaline ladder, the major cleavage sites generated by the endonuclease were identified as shown in FIG. 3B. Major cleavages sites generated by the endonuclease on nts 1705-1792 c-myc CRD (left panel in FIG. 3A), shown by solid lines, were at CA (nts 1727, 1742, 1768, 1771, 1775), UA (nts 1747, 1751, 1757, 1773) and UG (nts 1720, 1730). To further address the enzyme's specificity for those cleavage sites and to further identify other endonucleolytic cleavage sites on the CRD, we challenged the enzyme with nts 1705-1886 of c-myc CRD RNA. As shown in the right panel of FIG. 3A, the same cleavage sites, indicated by asterisks (nts 1742, 1747, 1751 and 1757), were generated by the endonuclease on both 1705-1792 and 1705-1886 RNA substrates. Cleavage sites at nts 1766, 1768, 1771, 1773 and 1775 generated on substrate 1705-1886 CRD RNA (right panel in FIG. 3A), were also generated on the substrate 1705-1792 CRD RNA (left panel in FIG. 3A). Additionally, the right panel in FIG. 3A shows that the endonuclease cleaved at CA sites at nts 1788, 1792, 1800 and 1808, further confirming that the enzyme cleaves some CA sites. FIG. 3B shows the summary of sites in CRD that are cleaved by the endonuclease.

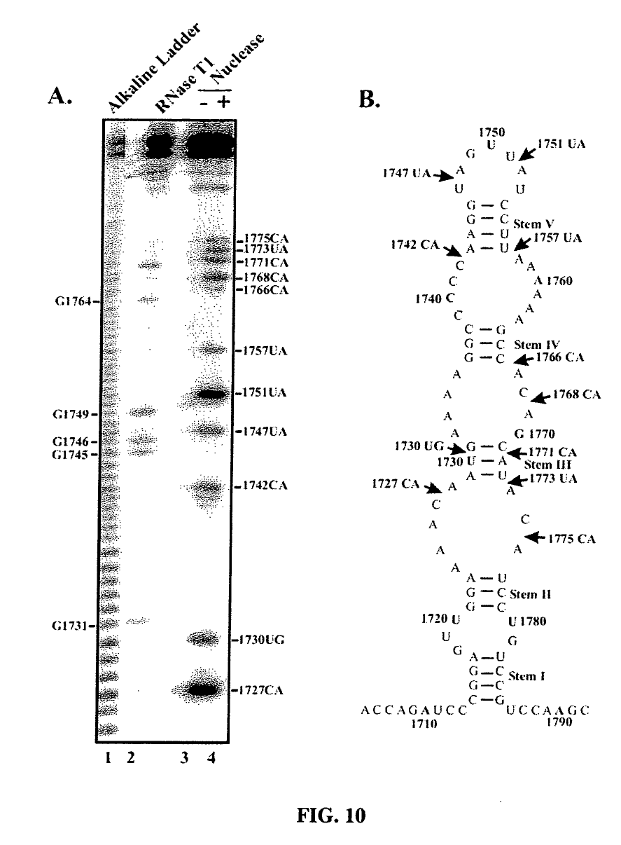

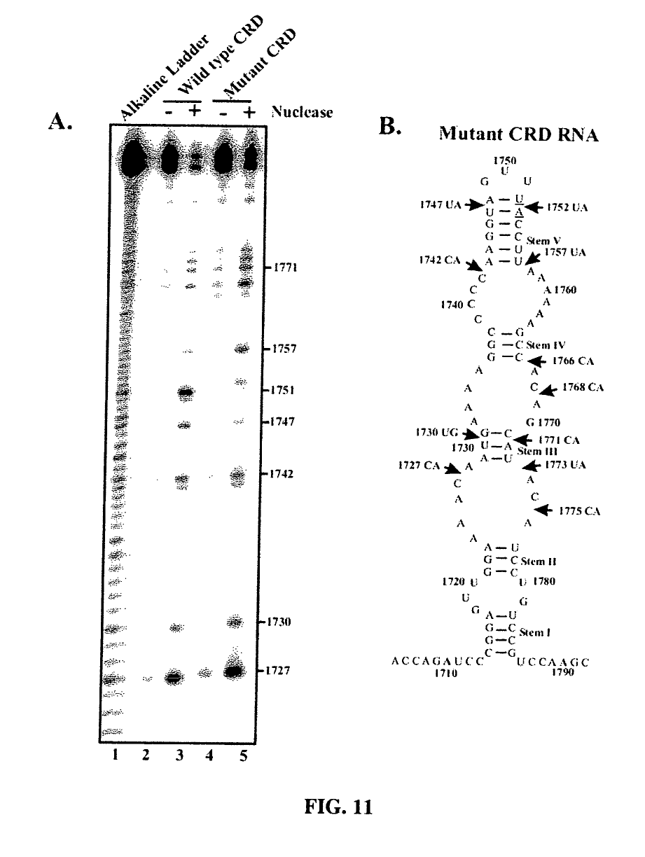

Further analysis of the cleavage sites indicated that the endoribonuclease cleaves in between UA, CA, UG dinucleotides in both single- and double-stranded RNA regions (FIGS. 10 and 11). It was also found that the endoribonuclease can cleave in between the CU dinucleotide, but only in the single-stranded conformation.

The Enzyme is an Endonuclease

A comparison of partial digests of the CRD probe with RNase T1 and alkaline digestion, was performed according to the method of Moine et al. (1998) RNA Structure and Function, pg 77-115, CSHL Press). The endonuclease was incubated with 88-nt CRD RNA (nts 1705-1792) that was ³²P-labeled at either the 5′ or 3′ terminus. The 5′-labeled substrate was cleaved by the endonuclease to generate four major distinct fragments which were easily observable on a 12% polyacrylamide gel. These four fragments were identified as nts 1742, 1747, 1751 and 1757, numbered 1-4 as shown in FIG. 4 (lanes 4 and 5). The 3′-labeled substrate was also cleaved by the enzyme to generate four distinct fragments which were observable on a 12% denaturing gel (lanes 9 and 10 in FIG. 4). Examination of these fragments (bands 1-4) (lanes 9 and 10) showed that they corresponded precisely to the four 5′-labeled degradation products (bands 1-4) (lanes 4 and 5) generated by the enzyme. These results lead us to conclude that the enzyme is an endonuclease. Analysis of the cleavage sites produced by the ribonuclease indicated that it preferentially cleaves phosphodiester bonds 3′ of U and 5′ of A (e.g., UU, UG, UA, CA).

The Endonuclease is a Protein-Only Enzyme

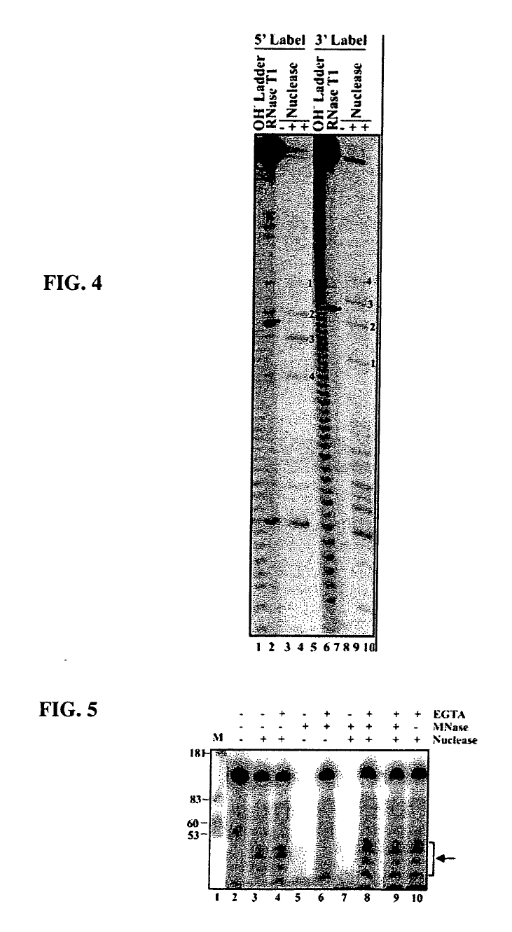

To address whether an RNA cofactor is required for activity by the endonuclease, the enzyme was treated with micrococcal nuclease as shown in the experiment described in FIG. 5. Lane 3 shows that degradation of ³²P-CRD RNA by the endonuclease using the standard endonuclease assay. Addition of 17 mM EGTA further enhanced the enzyme activity (lane 4). This is consistent with our findings that the endonuclease activity was increased in the presence of EDTA. Addition of 5×10⁻² units of micrococcal nuclease, either alone (lane 5), or in the presence of the endonuclease (lane 7), resulted in complete digestion of ³²P-CRD RNA. This degradation was prevented by co-addition of 17 mM EGTA (lanes 6 and 8). In lane 9, 3 units of the endonuclease were first incubated with micrococcal nuclease. 17 mM EGTA was then added to inactivate the micrococcal nuclease followed by incubation with ³²P-CRD RNA. Lane 10 is similar to lane 9, except that the endonuclease was incubated with water instead of micrococcal nuclease. Our results show the endonuclease remained active in the presence of EGTA, and that addition and pretreatment of micrococcal nuclease had no effect on the endonuclease activity. We conclude that the endonuclease does not require an RNA cofactor for activity and that catalytic RNA is not responsible for the nucleolytic activity observed in our assays.

The Enzyme Generates Product with 3′ Hydroxyl Group

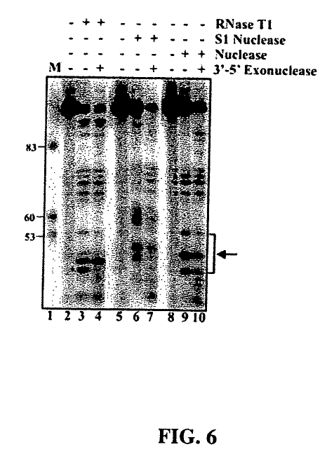

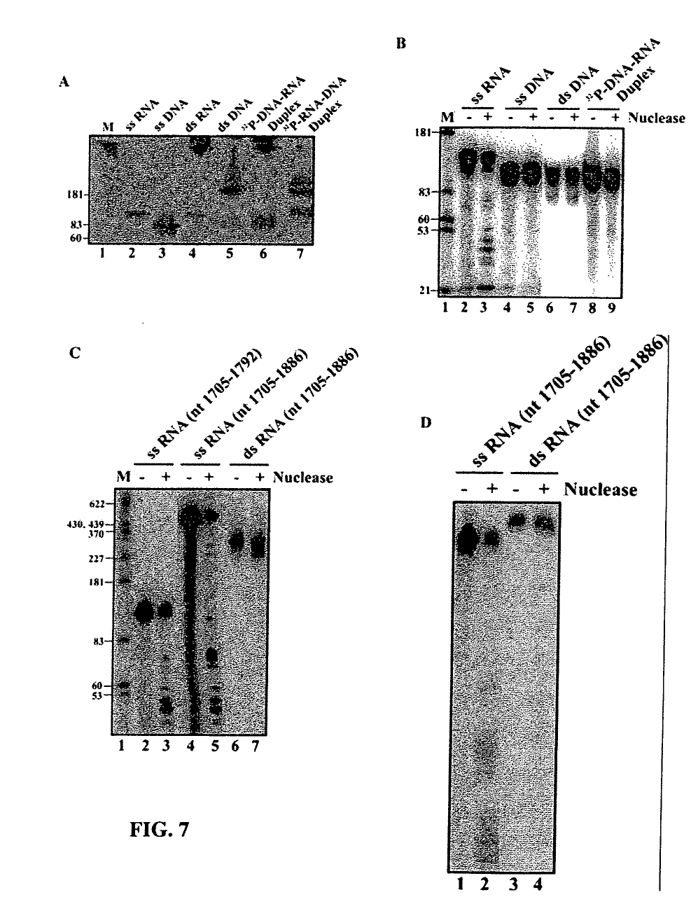

To determine whether the endonuclease generates products with a 3′ phosphate or a 3′ hydroxy group, the method described by Schoenberg and co-workers (Chernokalskaya et al., Nucleic Acids Res 25:735-742, 1997; Schoenberg and Cunningham, Methods, 17(1):60-73, 1999), in which digestion with snake venom exonuclease was used to differentiate between products with 3′ hydroxyls versus bearing 3′phosphates, was performed. FIG. 6 shows where 5′-labeled ³²P-CRD RNA was digested with RNase T1 to generate products with 3′ phosphates (lanes 3 and 4), S1 nuclease to generate products with 3′ hydroxyls (lanes 6 and 7), and the endonuclease (lanes 9 and 10). The products of these reactions were either loaded directly onto the gel without further treatment (lanes 3, 6 and 9), or treated with snake venom exonuclease prior to electrophoresis (lanes 4, 7 and 10). The snake venom exonuclease had no effect on most of the degradation products generated by RNase T1 cleavage (compare lane 4 to lane 3). One RNase T1 product (marked with an asterisk in lane 3) was degraded by the exonuclease (compare lane 4 to lane 3). The exonuclease was inert to the RNA substrate (compare lane 4 to lane 3), suggesting that there may be sub-population of substrates with 3′ phosphate groups. In contrast, most of the S1 nuclease degradation products (˜40-60 nts) were susceptible to degradation by the snake venom exonuclease, as evidence by the disappearance of fragments (compare lane 7 to lane 6). Four degradation products of ˜60-83 nts were distinctly generated only upon treatment with the exonuclease (compare lane 7 to lane 6). The ability of snake venom exonuclease to degrade the endonuclease-generated degradation products was investigated next. As shown in lane 9 (FIG. 6), the three typical decay products corresponding to cleavage sites 1720, 1727 and 1730 (shown by arrow) were generated by the endonuclease in addition to the generation of four decay products (shown by asterisks) corresponding to cleavage sites 1742, 1747, 1751 and 1757. The latter four decay products were not taken into consideration when assessing the nature of 3′ end RNA product generated by the endonuclease because these decay products were also produced by the exonuclease (compare lane 9 to lane 7). Thus, in evaluating the nature of 3′ end of RNA decay product generated by the endonuclease, we focused on the three typical decay products corresponding to cleavage sites 1720, 1727 and 1730. As shown in lane 10, upon treatment with the snake venom exonuclease, three endonuclease-generated decay products (shown by arrow) showed definitive degradation, suggestive of the presence of 3′ hydroxyl group for 3′-5′ exonucleolytic degradation by the exonuclease. Two separate experiments were conducted and similar results to FIG. 6 were obtained. Under the conditions conducted here, there were incomplete exonucleolytic digestions by the snake venom exonuclease on both S1 nuclease- (compare lane 7 to lane 6) and endonuclease-generated (compare lane 1.0 to lane 9) decay products.

We also performed another set of experiments using alkaline phosphatase. Internally ³²P-labeled 1705-1792 CRD RNA was first digested with the endonuclease followed by treatment with alkaline phophatase. When run on a 20% high resolution polyacrylamide/urea gel, the mobility shift of endonuclease- and S1 nuclease-generated degradation products were identical upon treatment with phosphatase. In contrast, there were greater mobility shifts of RNase T1-generated degradation products of similar sizes. This supports the conclusion that the endonuclease generates degradation products with 3′ hydroxyl groups.

The Enzyme Cleaves Single-Stranded RNAs and Double-Stranded RNA, but not DNAs or DNA-RNA Duplexes

The ability of the ribonuclease to cleave different substrates was analyzed (FIGS. 7A-D). We challenged the enzyme with single- and double-stranded nucleic acids, which correspond to the CRD sequences of c-myc mRNA. Double stranded c-myc RNA spanning nucleotides 1705-1792 was made by hybridizing uniformly ³²P-labeled sense transcript with unlabeled antisense transcript (Schoenberg and Cunningham, Methods, 17(1):60-73, 1999). Sense c-myc oligonucleotides spanning nucleotides 1705-1792 were purchased from QIAGEN and 5′ ³²P-labeled for use as single stranded DNA substrate. This single stranded DNA substrate was hybridized to its complementary antisense c-myc DNA (QIAGEN) to create the double stranded DNA substrate. Similarly, the RNA-DNA duplex was made by hybridizing the 5′ ³²P-labeled DNA with antisense RNA to c-myc spanning nucleotides 1705-1792.

To ensure that the correct substrates were used, we ran each nucleic acid sample on a 10% non-denaturing polyacrylamide gel followed by gel purification of the nucleic acid samples as shown in FIG. 7A. The asterisk on each lane shows the desired nucleic acid samples which were gel purified. Lane 2 shows the 5′-labeled 88-nt 1705-1792 CRD RNA and lane 3 shows the expected smaller fragment of 5′-labeled 88-nt 1705-1792 CRD DNA. Lane 4 shows the intact double-stranded RNA upon hybridization of 5′³²P-labeled 1705-1792 CRD RNA with excess antisense 1886-1705 CRD RNA. Also present in lane 4 is the single-stranded 5′³²P-labeled 1705-1792 CRD RNA which was either denatured or previously un-hybridized. Lane 5 shows the intact double-stranded DNA upon hybridization of 5′³²P-labeled 1705-1792 CRD DNA with excess antisense 1792-1713 CRD oligo. The single-stranded 5′³²P-labeled 1705-1792 CRD DNA was also present in small amounts indicating that parts of the double-stranded DNA was denatured or some of the single-stranded DNA was not previously hybridized. Two types of DNA-RNA duplexes were made as shown in lanes 6 and 7. Lane 6 shows the DNA-RNA duplex upon hybridization of 5′32P-labeled 1705-1792 CRD DNA to excess antisense 1886-1705 CRD RNA, and lane 7 shows the RNA-DNA duplex upon hybridization of 5′³²P-labeled 1705-1792 CRD RNA to excess antisense 1792-1713 CRD oligo. Traces of single-stranded 5′³²P-labeled DNA or RNA were also visible in these lanes for the same reasons described above. Nevertheless, only the desired nucleic acids were gel purified and tested for effects by the endonuclease.

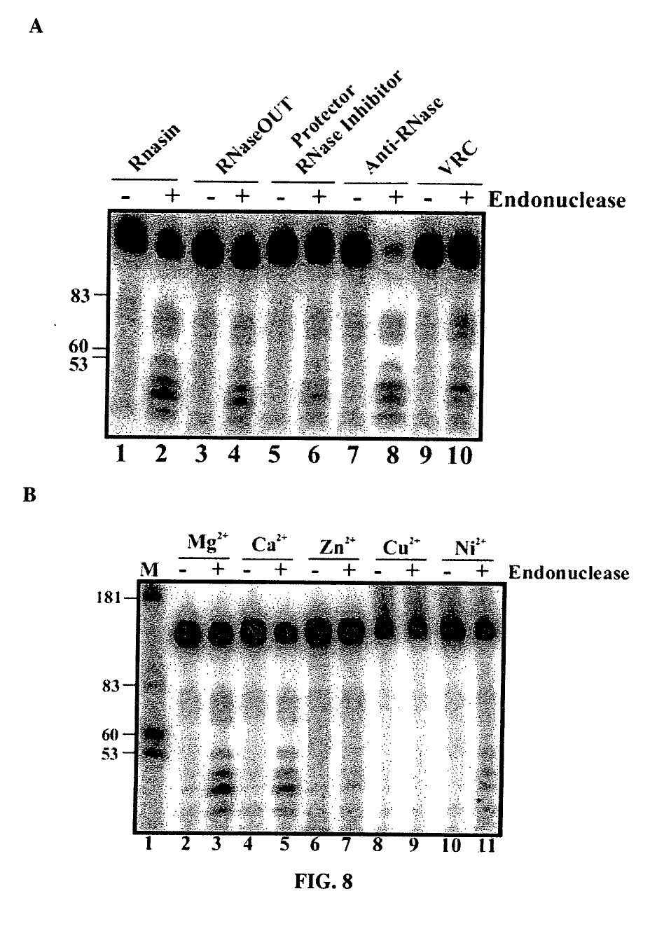

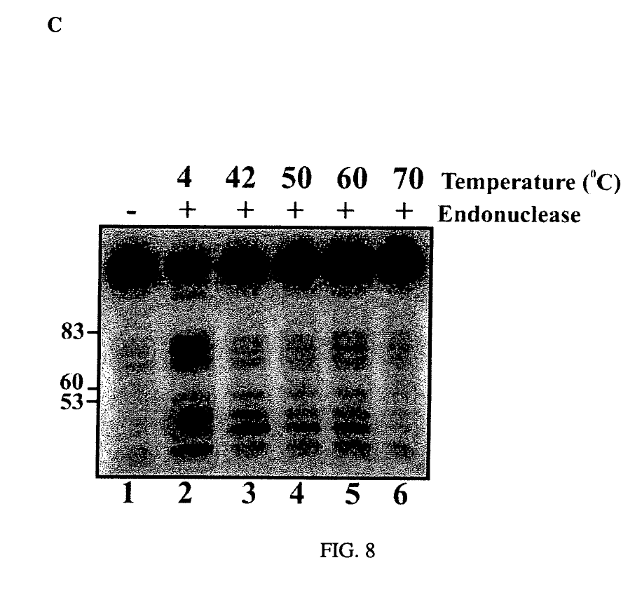

The endonuclease was challenged with gel purified nucleic acid substrates from FIG. 7A and samples were run on a 6% denaturing gel as shown in FIG. 7B. Lane 2 and 3 shows the typical three distinct products of ˜40-60 nts generated by the endonuclease on 5′³²P-labeled 1705-1792 CRD RNA. In contrast, the enzyme had no effect on the single stranded 5′³²P-labeled 1705-1792 CRD DNA (lanes 4 and 5) as well as the double-stranded DNA (lanes 5 and 6). Similarly, the endonuclease had no observable effect on DNA-RNA duplexes when either 5′³²P-labeled 1705-1792 CRD DNA (lanes 8 and 9) or 5′³²P-labeled 1705-1792 CRD RNA was used. These results were further confirmed in experiments where treated samples were run on 10% non-denaturing gel. To assess the possible double-stranded RNase activity of the endonuclease, we challenged the enzyme with double-stranded RNA made from complementary single-stranded RNA of identical length. 5′³²P-labeled 1705-1886 CRD RNA was hybridized with excess antisense 1886-1705 CRD RNA, and the double-stranded RNA was purified as in FIG. 7A. This double-stranded RNA was treated with the endonuclease and samples were run on either 6% denaturing gel (FIG. 7C) or 8% non-denaturing gel (FIG. 7D). The double-stranded RNA appeared to remain intact and have greater mobility (lanes 6 and 7) than its single-stranded counterparts of identical length (lanes 4 and 5) on a denaturing gel (FIG. 7C). This could be attributed to the shape of the double-stranded RNA molecule. On the denaturing gel, slightly faster migrating bands were observed from the double-stranded RNA upon treatment with the enzyme (lanes 6 and 7, FIG. 7C) which is indicative of ‘nibbling’ at the ends of RNA substrates. On the non-denaturing gel, the double-stranded RNA has slightly lower mobility than its single-stranded counterparts (compare lane 1 to 3, FIG. 7D), and upon treatment with the endonuclease, the double-stranded RNA was slightly degraded (lanes 3 and 4, FIG. 7D). This is consistent with the results in FIG. 7C of the possible ‘nibbling’ effect at the ends of RNA substrates by the enzyme.

From the above results, we conclude that the endonuclease can cleave both single-stranded and double-stranded RNAs, but not single- nor double-stranded DNAs, demonstrating that the enzyme is an RNA-specific endonuclease. Furthermore, it does not cleave RNA-DNA duplexes, suggesting that it does not possess RNase H-like activity. Thus, the results indicated that the ribonuclease cleaves phosphodiester bonds in non-base paired single stranded RNA sites, as well as double-stranded complementary RNA molecules, but does not cleave DNA molecules.

Sensitivity to Inhibitors, Metal Ions, and Temperature

The endonuclease was subjected to various inhibitors, metal ions, and temperatures and then examined for nucleolytic activity. The endonuclease was not inactivated by known RNase inhibitors such as vanadyl ribonucleoside complex, VRC (New England Biolabs), RNasin (Promega), RNaseOUT (Invitrogen), Protector RNase Inhibitor (Roche Diagnostics GmbH), and Anti-RNase (Ambion) (FIG. 8A), indicating that it is likely to be active under physiological conditions. Analysis of ribonuclease activity in the presence of metal ions indicated that it is active in the presence of Mg²⁺ and Ca²⁺, inactive in the presence of Cu²⁺, and has weak activity in the presence of Zn²⁺ and Ni²⁺ (FIG. 8B). The ribonuclease was active at temperatures up to at least 60° C. and could be inactivated at 70° C. (FIG. 8C), indicating that it is relatively stable for example at physiological temperatures or higher.

The Endonuclease Cleaves RNAs Other than c-myc mRNA

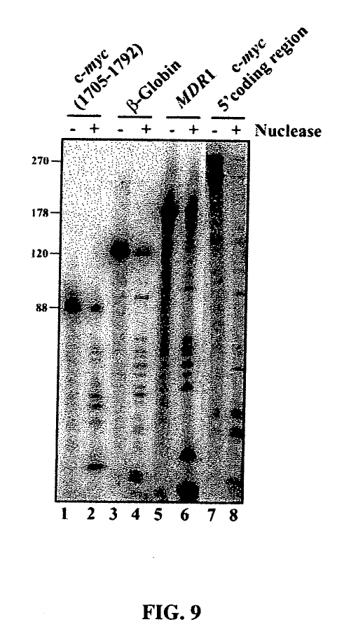

The endonuclease was challenged with four deproteinized 5′-³²P-RNA substrates as follows: c-myc 5′-CRD RNA (nts 1705-1792), β-globin RNA (nts 50-169), MDR1 RNA (nts 745-922), and nts 1-270 from the c-myc mRNA. As shown in FIG. 9, all RNAs tested were cleaved by the endonuclease to generate specific fragments. Judging from the identical cleavage sites generated by the endonuclease on nts 1705-1792 and 1705-1886 of c-myc RNA (FIG. 3A), and the specific fragments generated by the enzyme on other RNAs, we conclude that the endonuclease is not substrate-specific but rather a sequence-selective RNA endonuclease.

### EXAMPLE 2

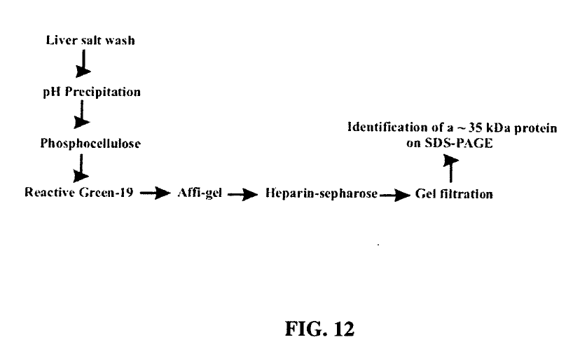

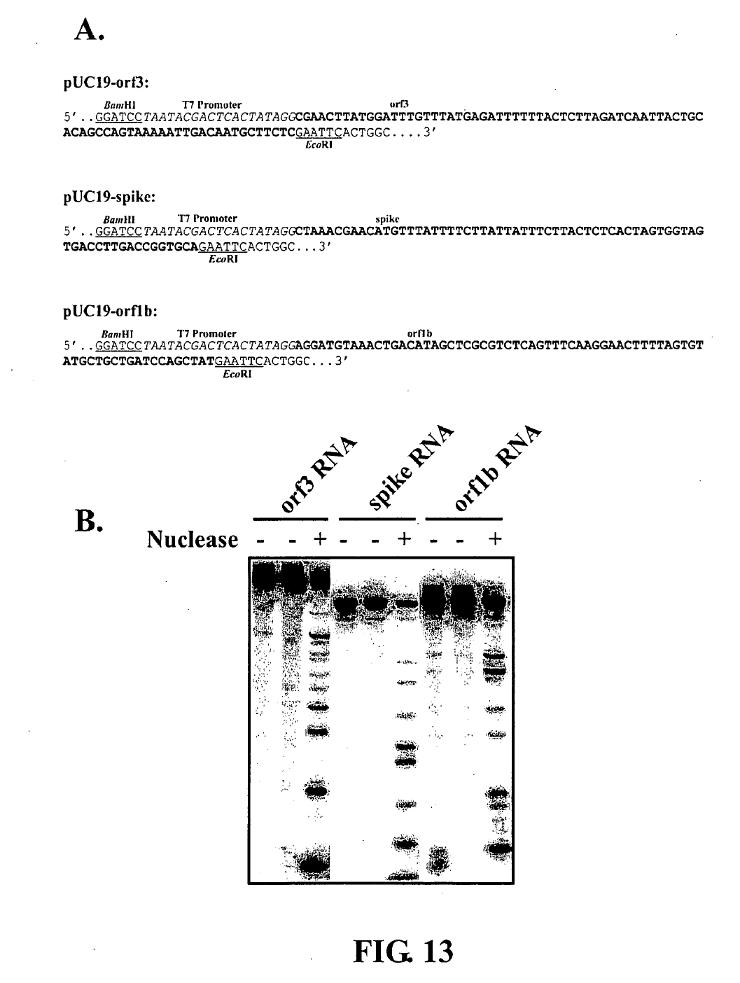

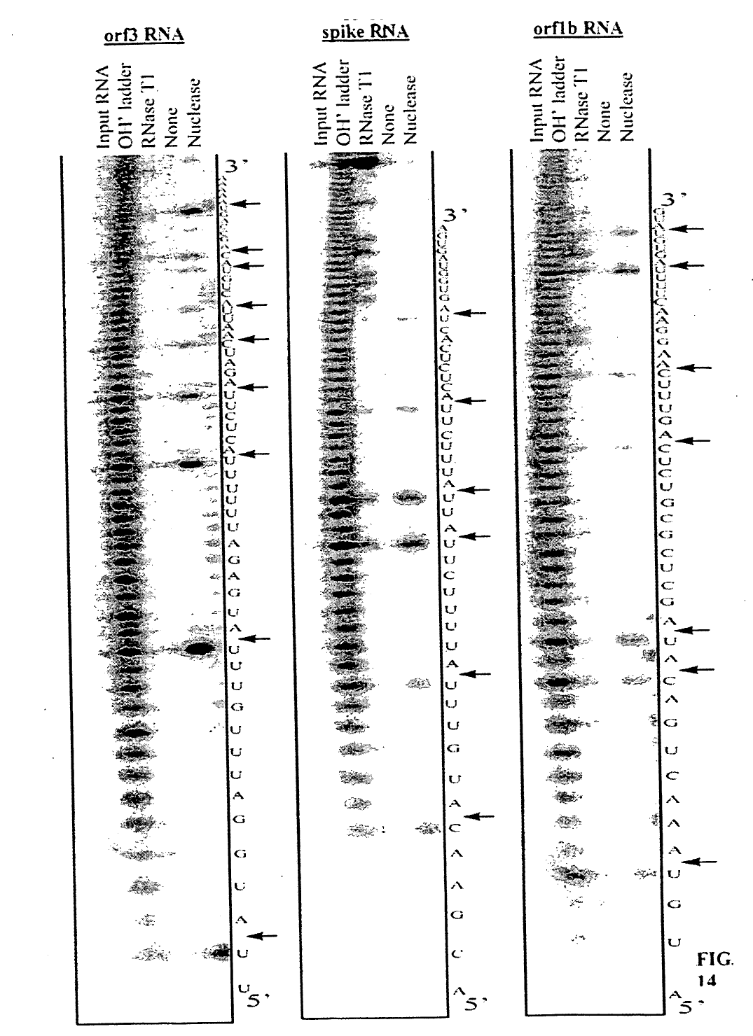

The endonuclease was prepared using a modified purification scheme in which the Reactive Blue-4 step was removed and a Gel Filtration step was added (FIG. 12). The ability of the endonuclease to cleave orf3, S and orf1b RNAs of the SARS coronavirus, which are important for the virus replication and life cycle, was tested. We first constructed pUC19-orf3, pUC19-spike, and pUC19-orf1b (FIG. 13A) and then transcribed the corresponding RNA (FIG. 13B). The results indicated that the endoribonuclease can cleave in between UA and CA dinucleotides within orf3, Spike, and orf1b RNAs (FIG. 14).

### Other Embodiments

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to”, and the word “comprises” has a corresponding meaning. Citation of references herein shall not be construed as an admission that such references are prior art to the present invention. All publications are incorporated herein by reference as if each individual publication were specifically and individually indicated to be incorporated by reference herein and as though fully set forth herein. The invention includes all embodiments and variations substantially as hereinbefore described and with reference to the examples and drawings.

What is claimed is:

1. A substantially pure endoribonuclease, wherein the endoribonuclease has a molecular weight of about 34 to about 48 KDa on a 12% denaturing SDS polyacrylamide gel and is capable of preferentially cleaving a RNA molecule at sites 3′ of uracil residues or sites 5′ of adenine residues.

2. The endoribonuclease of claim 1, wherein the endoribonuclease is thermostable to a temperature of about 70° C.

3. The endoribonuclease of claim 1, wherein the endoribonuclease is active between a temperature of about 4° C. to about 60° C.

4. The endoribonuclease of claim 1, wherein the endoribonuclease is inactive in the presence of Cu²⁺.

5. The endoribonuclease of claim 1, wherein the endoribonuclease is active in the presence of Mg²⁺, Ca²⁺, Zn²⁺ or Ni²⁺.

6. The endoribonuclease of claim 1, wherein the endoribonuclease is active in the presence of RNAase inhibitors selected from the group consisting of one or more of vanadyl ribonucleoside complex (VRC), RNasin, RNaseOUT, Protector RNase Inhibitor, and Anti-RNase

7. The endoribonuclease of claim 1, wherein the endoribonuclease is further capable of cleaving a RNA molecule at sites 3′ of cytosine residues or sites 5′ of guanine residues.

8. The endoribonuclease of claim 1, wherein the endoribonuclease is capable of cleaving one or more of a sequence selected from the group consisting of UA, CA, UG, and CU.

9. The endoribonuclease of claim 1, wherein the endoribonuclease is capable of cleaving non-base paired RNA, single stranded RNA, or double stranded RNA.

10. The endoribonuclease of claim 1, wherein the endoribonuclease is capable of cleaving c-myc mRNA, SARS orf3 RNA, SARS spike RNA, or SARS orf1b RNA.

11. The endoribonuclease of claim 1, wherein the endoribonuclease is a mammalian endoribonuclease.

12. The endoribonuclease of claim 11, wherein the mammalian endoribonuclease is a rat endoribonuclease or a human endoribonuclease.

13. A method of treating or preventing a disorder associated with aberrant RNA expression, viral RNA expression, or c-myc RNA expression, the method comprising administering the endoribonuclease of claim 1.

14. The method of claim 13 wherein the c-myc RNA is CRD RNA.

15. The method of claim 13 wherein the endoribonuclease comprises a fusion polypeptide.

16. The method of claim 15 wherein the fusion polypeptide comprises a targeting molecule capable of targeting the endoribonuclease to a virus-infected cell or a cancer cell.

17. A method of cleaving a RNA molecule comprising contacting the RNA molecule with the endoribonuclease of claim 1 under conditions in which the endoribonuclease is active, wherein the endoribonuclease cleaves the RNA molecule.

18. A method of screening a compound for modulating endoribonuclease activity comprising
a) providing the endoribonuclease of claim 1;
b) providing a RNA molecule substrate capable of being cleaved by the endoribonuclease;
c) providing a test compound; and
d) determining whether the test compound modulates the cleavage of the RNA molecule substrate.

19. The method of claim 18, wherein the method is carried out within a cell or carried out using a cell free system.

20. A kit comprising the endoribonuclease of claim 1, together with instructions for use of the endoribonuclease.


SEQUENCE LISTING (10 sequences)

SEQ ID NO: 1 - RNA, 108 nt, Homo sapiens
  nt 1705-1812 of c-myc CRD RNA
accagaucccggaguuggaaaacaaugaaaaggcccccaagguaguuauccuuaaaaaagccacagcauacauccuguccguccaagcagaggagcaaaagcucauuu

SEQ ID NO: 2 - RNA, 88 nt, Homo sapiens
  1705-1792 CRD RNA
accagaucccggaguuggaaaacaaugaaaaggcccccaagguaguuauccuuaaaaaagccacagcauacauccuguccguccaagc

SEQ ID NO: 3 - RNA, 88 nt, Homo sapiens
  MUTANT 1705-1792 CRD RNA
accagaucccggaguuggaaaacaaugaaaaggcccccaagguaguuuaccuuaaaaaagccacagcauacauccuguccguccaagc

SEQ ID NO: 4 - DNA, 80 bp, SARS Coronavirus
  orf 3
cgaacttatggatttgtttatgagattttttactcttagatcaattactgcacagccagtaaaaattgacaatgcttctc

SEQ ID NO: 5 - DNA, 68 bp, SARS Coronavirus
  spike
ctaaacgaacatgtttattttcttattatttcttactctcactagtggtagtgaccttgaccggtgca

SEQ ID NO: 6 - DNA, 70 bp, SARS Coronavirus
  orf1b
aggatgtaaactgacatagctcgcgtctcagtttcaaggaacttttagtgtatgctgctgatccagctat

SEQ ID NO: 7 - DNA, 45 bp, Homo sapiens
  FUP Primer
ctcggatccatttaggtgacactatagaccagatcccggagttgg

SEQ ID NO: 8 - DNA, 27 bp, Homo sapiens
  RUP Primer
ctcgaattcgcttggacggacaggatg

SEQ ID NO: 9 - DNA, 27 bp, Homo sapiens
  F1705-1722 primer
ctcggatccaccagatcccggagttgg

SEQ ID NO: 10 - DNA, 28 bp, Homo sapiens
  R1869-1886 primer
cgacaagcttcgcacaagagttccgtag